(19) United States
(12) Patent Application Publication
(10) Pub. No.: US 2024/0108679 A1
(43) Pub. Date: Apr. 4, 2024

(54) Composition and manufacturing processes of a toxicity free botanical drug for relief from breathing difficulties

(71) Applicants: ASHOK KUMAR, SCOTTSDALE, AZ (US); PRAKRATI CHIRAG ACHARYA, EL PASO, TX (US); PRITI KUMAR, ALIGARH (IN); ADITI KUMAR, SCOTTSDALE, AZ (US)

(72) Inventors: ASHOK KUMAR, SCOTTSDALE, AZ (US); PRAKRATI CHIRAG ACHARYA, EL PASO, TX (US); PRITI KUMAR, ALIGARH (IN); ADITI KUMAR, SCOTTSDALE, AZ (US)

(21) Appl. No.: 17/958,322

(22) Filed: Oct. 1, 2022

(51) Int. Cl. A61K 36/73; A61K 9/00; A61K 9/14; A61K 36/9064

(52) U.S. Cl. CPC A61K 36/73; A61K 9/0019; A61K 9/0056; A61K 9/14; A61K 36/9064

(57) ABSTRACT

This invention discloses composition and manufacturing processes of a toxicity free botanical drug formulation for relief from breathing difficulties. This botanical drug is manufactured from plants Syzygium aromaticum or component(s) thereof, plant Elettaria cardamomum or component(s) thereof and sweeteners or component(s) thereof. This botanical drug was administered to human volunteers by buccal, supralingual, sublingual and oral routes in effective amount for an effective time-period. Effective amount and effective time of administration varies from one human volunteer to another depending upon type and severity of breathing difficulties.

### CROSS REFERENCE TO RELATED APPLICATIONS (IF ANY)

Not applicable.

### STATEMENT OF FEDERALLY SPONSORED RESEARCH OR DEVELOPMENT (IF ANY)

Not applicable.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

Not applicable.

### REFERENCE TO A “SEQUENCE LISTING” A TABLE OR A COMPUTER PROGRAM LISTING APPENDIX SUBMITTED ON A COMPACT DISC AND AN INCORPORATION BY REFERENCE OF THE MATERIAL ON THE COMPACT DISC. THE TOTAL NUMBER OF COMPACT DISC INCLUDING DUPLICATES AND THE FILES ON EACH COMPACT DISC SHALL BE SPECIFIED

Not applicable.

### BACKGROUND OF THE INVENTION

### Description of the Prior Art

Patients suffering from breathing difficulties experience discomfort while breathing. Specific non-limiting examples of such diseases are Asthma, Chronic Obstructive Pulmonary Disease (COPD), Bronchitis, Vocal Cord Dysfunction (VCD), Interstitial lung diseases etc. But this invention is not limited to breathing difficulties experienced by patients suffering from Asthma, Chronic Obstructive Pulmonary Disease (COPD), Bronchitis, Vocal Cord Dysfunction (VCD), Interstitial lung diseases. Breathing difficulties experienced by patients of other diseases are also included in this invention.

Some of the chemical pharmaceuticals having varying degree of adverse effects used by patients suffering from breathing difficulties are mentioned below.

### Long Term Control Medications:

Long-acting beta agonists, Leukotriene modifiers (Montelukast, Zafirlukast, Zileuton), corticosteroids (Fluticasone, Budesonide, Mometasone, Beclomethasone, Ciclesonide), Theophylline etc. are long term control medications required to be taken regularly to control and prevent breathing difficulties.

### Quick-Relief Medications:

Short-acting beta agonists (Albuterol, Levalbuterol), Ipratropium, corticosteroids (Prednisone, Methylprednisolone) etc. are quick-relief medications used for rapid short-term relief from breathing difficulties.

### Drawbacks of Prior Art

Long term control medications such as long-acting beta agonists, leukotriene modifiers, corticosteroids, theophylline taken regularly to control and prevent breathing difficulties cause side effects such as insomnia, gastroesophageal reflux, agitation, aggression, hallucinations, depression, suicidal thinking etc.

Quick-relief medications such as short-acting beta agonists, Ipratropium, corticosteroids etc. to be taken for rapid short-term relief from breathing difficulties cause side effects such as palpitations, jitteriness, cataract, osteoporosis, muscle weakness, high blood pressure, decreased resistance to infections, reduced growth in children etc.

### Solution to Drawbacks:

Toxicity free Botanical drug described in this invention provides relief from breathing difficulties when an effective amount is administered to the patients for effective time-periods.

### BRIEF SUMMARY OF THE INVENTION

### Objects of the Invention

The main object of this invention is to develop composition of a toxicity free botanical drug formulation for relief from breathing difficulties.

Further object of this invention is to develop composition and manufacturing processes of this toxicity free botanical drug formulation.

Another object of this invention is to develop methods of treatment of patients with this toxicity free botanical drug formulation so that they get relief from breathing difficulties.

### Field of Invention

This invention relates to composition and manufacturing process(es) of a toxicity free botanical drug formulation from plant Syzygium aromaticum or component(s) thereof, plant Elettaria cardamomum or component(s) thereof and sweeteners or component(s) thereof for relief from breathing difficulties.

### BRIEF DESCRIPTION OF THE INVENTION

This invention describes a toxicity free botanical drug, its composition and manufacturing process(es) which provides relief from breathing difficulties when an effective amount is administered to patients for effective time-period. This drug comprises of about 0.01% to about 99.999% of Syzygium aromaticum or component(s) thereof, about 0.001% to about 40% of Elettaria cardamomum or component(s) thereof and about 0.001% to about 95% of sweeteners or component(s) thereof.

### Toxicology

This botanical drug was administered to human volunteers for long term, but no toxic effects were observed.

### BRIEF DESCRIPTION OF THE SEVERAL VIEWS OF THE DRAWING (IF ANY)

Not applicable.

### DETAILED DESCRIPTION OF THE INVENTION

This invention discloses a toxicity free botanical drug and its manufacturing process(es) for relief from breathing difficulties. This botanical drug is manufactured from plants Syzygium aromaticum or component(s) thereof, plant Elettaria cardamomum or component(s) thereof and sweeteners or component(s) thereof. This botanical drug is administered to human volunteers in a defined manner. In one embodiment volunteers were healthy in another embodiment volunteers were patients having confirmed diagnosis of breathing difficulties. These human volunteers were administered an effective amount of botanical drug and kept under observation.

### Explanation of the Terms

### Botanical Drug Formulation:

This invention discloses a toxicity free botanical drug, comprising of about 0.01% to about 99.999% of Syzygium aromaticum or component(s) thereof, about 0.001% to about 40% of Elettaria cardamomum or component(s) thereof and about 0.001% to about 95% of sweeteners or component(s) thereof. In this invention addition of plant Elettaria cardamomum or component(s) thereof enhances the action of plant Syzygium aromaticum or component(s) thereof. Additionally, in this invention addition of sweeteners or component(s) thereof enhances the action of plant Syzygium aromaticum or component(s) thereof and Elettaria cardamomum or component(s) thereof resulting in increased airflow to the lungs in human volunteers suffering from breathing difficulties.

In this invention component(s) denotes to any component(s) from plants Syzygium aromaticum, Elettaria cardamomum and sweeteners. Specific non-limiting examples of components are extract(s), fraction(s), isolated and/or purified molecule(s), phytochemical(s), synthetic analogue of phytochemical(s) etc.

In this invention plants Syzygium aromaticum, Elettaria cardamomum and sweeteners include, but not limited to, any one or more than one or any other or every variety of plants Syzygium aromaticum, Elettaria cardamomum and sweeteners. In addition to this, plants Syzygium aromaticum, Elettaria cardamomum and sweeteners include, but not limited to, any one or more than one or every part of plants Syzygium aromaticum, Elettaria cardamomum and sweeteners including but not limited to be fresh, dehydrated or preserved in some manner.

In this invention botanical drug formulation can be in any form. Specific non-limiting examples of forms are solid, semisolid, liquid, suspension, paste, powder, tablets, pills, capsules etc. In this invention botanical drug can be given alone or in combination with other additive(s) including but not limited to herb(s) or component(s) thereof, any other biological material(s) or component(s) thereof, any other chemical(s), phytochemical(s), compound(s), mineral(s) etc.

Syzygium aromaticum:

Syzygium aromaticum is 8-12 meters tall evergreen tree with large leaves and crimson flowers in family Myrtaceae cultivated in Indonesia, India, Pakistan, Madagascar, Sri Lanka etc. Aromatic flower buds of Syzygium aromaticum commonly known as cloves are used as spice and in cigarette called kretek. It is also used in traditional medicine for toothache and other types of pain. Syzygium aromaticum has been used for food preservation and possesses various pharmacological activities (Batiha G E et al., Biomolecules. 2020 Jan. 30; 10(2). pii: E202. doi: 10.3390/biom10020202) such as anti-inflammatory (Lee H H et al. J Nat Prod. 2016; 79(4):711-20), anti-platelet (Srivastava K C., Prostaglandins Leukot Essent Fatty Acids. 1993; 48(5):363-72) etc.

Elettaria cardamomum:

Elettaria cardamomum is 2-4 meters tall pungent, aromatic, herbaceous, perennial plant with 40-60 cm long pointed tip leaves and white to lilac or pale violet flowers in the Zingiberaceae family native to Indian subcontinent and tropical regions. Fruits are yellow-green 1-2 cm long three-sided pod containing several seeds. Elettaria cardamomum are used in cuisines and possesses various activities such as anti-hypercholesterolemic (Nagashree S. et. al., J Sci Food Agric. 2017; 3204-3210), insecticidal (Al Dawsari, M M., Saudi J Biol Sci. 2020; 195-201), reduction in serum triglyceride (Esfahani, S P., et. al. Phytother Res. 2020; 475-485) etc.

### Sweeteners:

Sweeteners used are sugar-based and sugar substitute. Specific non-limiting examples of sugar-based sweeteners are honey, sucrose, glucose, fructose etc. Specific non-limiting examples of sugar substitute sweeteners are aspartame, sucralose, neotame, saccharin, stevia, sorbitol, xylitol, lactitol etc. Traditionally sweeteners are used as food.

### Toxicity Free:

This denotes a botanical drug formulation which is toxicity free on long term use even for years altogether. Slight staining of tongue (can be cleaned by tongue cleaner) and slight irritation on the tongue reported by some volunteers.

### Human Volunteers:

Human volunteers in the age range of 5 to 85 years were administered with this toxicity free botanical drug. Specific, non-limiting example of human volunteers are in one embodiment they were perfectly healthy in another embodiment they were patients suffering from breathing difficulties.

### Administration of the Botanical Drug in a Defined Manner:

Human volunteers are advised to take 25-5000 mgs of botanical drug when so ever they experience breathing difficulty. Specific non-limiting examples of breathing difficulties are shortness of breath, cough, bronchial spasms etc.

Routes of administration of the botanical drug include, but not limited to transmucosal, sublingual, supralingual, translingual, buccal, oral, topical, trans-dermal, intramuscular, intravenous, subcutaneous, intradermal, intranasal, topical, rectal, intraperitoneal or any other route which can be used for rapid drug absorption. Specific, non-limiting examples of routes of administration are as follows
Buccal: Insert botanical drug between cheek and gum close mouth and hold against cheek and allow it to dissolve slowly for absorption.
Supralingual: Put botanical drug on top of the tongue close mouth and allow it to dissolve slowly for absorption.
Sublingual: Put botanical drug under the tongue close mouth and allow it to dissolve slowly for absorption.
Patients were kept under observations. Botanical drug provides relief from difficulty in breathing within a few minutes.

### Effective Amount of the Botanical Drug:

A therapeutically effective amount (25-5000 mgs) is an amount sufficient to provide relief from breathing difficulties. Effective amount may be administered when so ever patient experience breathing difficulty—specific, non-limiting examples are such as about once in a minute, once in an hour, four times in an hour, once in a day, twice in a day, four times in a day, once in a week, twice in a week etc. Effective amount is varied from one patient to another depending upon type and severity of breathing difficulties.

### Effective Time-Period:

Effective time-period is the time sufficient for getting relief from breathing difficulties. Effective time-period varies from few second to several minutes. Effective time-period varies from one patient to another depending upon type and severity of breathing difficulties.

### Follow Up and Observation:

Every human volunteer, both healthy and patients suffering from breathing difficulties were advised to come to clinic and checked by the physician. Observations of volunteers and physicians were duly recorded, and more botanical drug was given to every volunteer for administration.

Relief from Breathing Difficulties:

Relief from breathing difficulties denotes increased airflow to the lungs in human volunteers suffering from breathing difficulties. Specific, non-limiting examples of relief from breathing difficulties are as follows,
After putting this botanical drug on or under the tongue patients no more feel shortness of breath.
After putting this botanical drug on or under the tongue patients no more feel cough etc.

### Breathing Difficulties:

Breathing difficulties describe discomfort during breathing and caused by different lung and heart conditions such as asthma, COPD, Vocal Cord Dysfunction (VCD), Pneumonia, allergies, viral infections etc. Novelty of this invention is that it discloses a toxicity free botanical drug formulation and its manufacturing process(es) for relief from breathing difficulties. This invention also discloses the method of treatment of patients suffering from breathing difficulties. In this invention three routes of inoculation were used, and these three routes are equally effective in providing relief from breathing difficulties. In some patients supralingual route was used but in other sublingual route was used while in other buccal route was used and some used all three routes. But scope of present invention should not be limited to supralingual, sublingual and buccal routes only. Specific, non-limiting examples of treatment of human volunteers (patients) suffering with breathing difficulties are as follows. But scope of present invention should not be limited to these only.

### Example 1

Relief from Breathing Difficulties in Patients Suffering from Asthma:

Asthma is a chronic inflammatory lung disease in which bronchi are reversibly narrowed. Patients suffering from Asthma experience breathing difficulties such as cough, shortness of breath etc. This botanical drug provides relief from breathing difficulties within few minutes as evidenced by spirometry also.

### Example 2

Relief from Breathing Difficulties in Patients Suffering from Chronic Obstructive Pulmonary Disease (COPD):

Chronic Obstructive Pulmonary Disease (COPD) is a lung disease that causes obstructed airflow in lungs and patients experience breathing difficulties. This botanical drug provides relief from breathing difficulties within few minutes as evidenced by spirometry also.

### Example 3

Relief from Breathing Difficulties in Patients Suffering from Bronchitis:

In both acute and chronic bronchitis bronchial tubes gets inflamed. Patients suffering from bronchitis experience breathing difficulties such as cough, shortness of breath etc. This botanical drug provides relief from breathing difficulties within few minutes as evidenced by spirometry also.

### Example 4

Relief from Breathing Difficulties in Patients Suffering from Interstitial Lung Disease (ILD):

Interstitial lung diseases (ILD) are chronic inflammatory lung diseases. Patients suffering from Interstitial lung diseases experience breathing difficulties such as cough, shortness of breath etc. This botanical drug provides relief from breathing difficulties within few minutes.

### Example 5

Relief from Breathing Difficulties in Patient Suffering from Vocal Cord Dysfunction (VCD):

Vocal cord dysfunction (VCD) also called as laryngeal dysfunction, laryngeal obstruction, paradoxical vocal cord movement disorder is abnormal closing of vocal cords during breathing. Patients suffering from Vocal cord dysfunction (VCD) experience breathing difficulties, cough, wheezing, throat tightness, hoarseness, shortness of breath etc. This botanical drug provides relief from breathing difficulties within few minutes.

1. A toxicity free composition for relief from breathing difficulties in a human in need thereof consisting essentially of therapeutically effective amounts of Syzygium aromaticum or component(s) thereof, Elettaria cardamomum or component(s) thereof, sweeteners or component(s) thereof and at least one excipient.

2. The composition of claim 1, wherein said Syzygium aromaticum or component(s) thereof is present in an amount of about 0.01% to about 99.999%, said Elettaria cardamomum or component(s) thereof is present in an amount of about 0.001% to about 40% and said sweeteners or component(s) thereof is present in an amount of about 0.001% to about 95% of the composition.

3. The composition of claim 1, wherein said composition is formulated to be administered orally, buccally, supralingually, sublingually, intramuscularly, intravenously, subcutaneously, intradermally, topically, transdermally, rectally or intraperitoneally and to be in any form such as solid, semi-solid, liquid, suspension, paste, pill, capsule, tablet, or powder.
